(19) United States
(12) Patent Application Publication
(10) Pub. No.: US 2023/0338257 A1
(43) Pub. Date: Oct. 26, 2023

(54) ANHYDROUS CONCENTRATED HAIR CARE COMPOSITIONS IN THE FORM OF A PASTE

(71) Applicant: EVERIST Inc., Etobicoke (CA)

(72) Inventors: Mandana AREFMANESH, Etobicoke (CA); La Chae HOOD, Toronto (CA); Sharon ROBERTSON, Toronto (CA); Jayme JENKINS, Etobicoke (CA); Jessica STEVENSON, Etobicoke (CA)

(21) Appl. No.: 18/142,497

(22) Filed: May 2, 2023

Related U.S. Application Data
(63) Continuation of application No. PCT/CA2021/051596, filed on Nov. 9, 2021.
(60) Provisional application No. 63/111,497, filed on Nov. 9, 2020.

(51) Int. Cl. A61K 8/44; A61K 8/46; A61K 8/41; A61Q 5/02; A61Q 5/12; A61K 8/34; A61K 8/9794; A61K 8/92; A61K 8/02; A61K 8/37

(52) U.S. Cl. CPC A61K 8/416; A61K 8/0216; A61K 8/345; A61K 8/375; A61K 8/44; A61K 8/466; A61K 8/92; A61K 8/9794; A61Q 5/02; A61Q 5/12; A61K 2800/31; A61K 2800/48

(57) ABSTRACT

The disclosure relates to cosmetic compositions, particularly concentrated hair care compositions in the form of a paste for shampooing and/or conditioning the hair. In one aspect, a shampoo composition is provided, wherein the shampoo composition is in the form of a paste; the shampoo composition comprises at least one surfactant, at least one organic solvent and optionally at least one thickener and/or at least one emollient, wherein the shampoo composition is free of, or substantially free of, added water. In another aspect, a hair conditioner is provided, wherein the conditioner composition is in the form of a paste; the conditioner composition comprises at least one thickener, at least one organic solvent, at least one emulsifier and rheology modifier, at least one emollient and at least one conditioning agent, wherein the conditioner composition is free of, or substantially free of, added water.

### CROSS-REFERENCE TO RELATED APPLICATIONS

The present application is a continuation of PCT Application No. PCT/CA2021/051596 filed on Nov. 9, 2021, which claims priority from U.S. Provisional Application No. 63/111,497 filed on Nov. 9, 2020, both incorporated herein by reference in their entireties.

### TECHNICAL FIELD

The following relates to cosmetic compositions, particularly concentrated cosmetic compositions for shampooing, cleansing, washing, moisturizing, conditioning, and/or exfoliating the hair or skin.

### BACKGROUND

Commonly available body and haircare products and compositions such as shampoos, body washes, hair masks, and conditioner products are typically in the form of viscous liquid compositions. Such products often contain significant quantities of liquid, such as water (usually 70-90% water), leading to dilution of the active ingredients and, in turn, relatively large volumes thereof being required for each use. Furthermore, containers for these products tend to be relatively large, single-use plastic containers to accommodate for the large volumes required to achieve a desirable number of uses per container leading to more plastic waste. Such containers can be considerably heavy and thus inefficient for transport and leave a larger carbon footprint. They are also not travel-friendly due to liquid restrictions on flights, the size and weight of the bottles, high likelihood of leakage, etc. Further, traditional formulas tend to use harsh surfactants such as sulfates and use other synthetic, for example petrochemical, ingredients that increase toxicity in our bodies and the environment. Preservatives, in particular, can have negative implications for human and environmental health and they are most necessary in cosmetic formulations containing water as water activity is what supports bacteria growth.

To address the above drawbacks, solid (or “dry”) personal care products, such as shampoo products, have been developed which contain surfactants and other actives at a higher concentration, and are typically more compact and leave a smaller carbon footprint and require significantly less packaging. A user is more likely to use a smaller amount of these concentrated products, reducing waste overall. Such solid compositions (bars, powders, tabs), however, are quite far from user’s current routine with liquid products and thus can be a barrier from transitioning to more eco-friendly options. They also tend to contain sulfates and other petrochemical derived ingredients that can be environmentally unfriendly and also harsh on a user’s skin or hair. They are often in the form of shampoo or conditioner bars, which can be messy and melt away in the shower when exposed to water. On the other hand, loose powder forms, can be difficult to use as they proof and clump when exposed to moisture in the shower. As both options (and many other options including shampoo ‘tabs’, ‘pods’ and ‘sheets’) are very different from the customary shampoo experience, the customer is required to change their behavior significantly in order to adopt these products into their routine. A waterless, water-activated paste format is very close to the traditional shampoo experience (easy to water-activate, does not need to dissolve/de-clump, not messy, etc.) and provides the eco, portability and convenience benefits outlined above.

In view of the foregoing, it is desirable to develop an improved concentrated cosmetic composition.

### SUMMARY

In one aspect, provided is a composition, for example, a shampoo composition and body wash composition, in the form of a paste, the composition comprising: at least one surfactant in an amount of 15 wt% to about 48 wt%; at least one thickener in an amount of about 0 wt% to about 10 wt%; at least one organic solvent in an amount of 25 wt% to about 60 wt%; and at least one emollient in an amount of 0 wt% to 15 wt%. In one embodiment, the shampoo composition may comprise at least one conditioning agent in an amount of about 0 wt% to about 30 wt%; wherein the shampoo composition is free of, or substantially free of, water.

In an implementation, the at least one surfactant is an anionic surfactant.

In another implementation, the at least one organic solvent is a polyol.

In yet another implementation, the at least one organic solvent is glycerin.

In yet another implementation, the at least one organic solvent is glycerin and aloe vera gel.

In yet another implementation, the at least one surfactant is sodium cocoyl isethionate and/or sodium lauroyl glutamate.

In yet another implementation, the at least one thickener is lysolecithin, sclerotium gum, xanthan gum, and/or pullulan.

In yet another implementation, the at least one emollient is glyceryl caprylate/caprate.

In yet another implementation, the composition further comprises propanediol.

In yet another implementation, the composition further comprises at least one oil, for example, Amla oil.

In yet another implementation, the composition further comprises a conditioning agent, for example, polyglyceryl-3 betainate acetate.

In yet another implementation, the composition further comprises dead sea salt coarse.

In another aspect, there is provided a composition, for example a conditioner composition and hair mask composition, in the form of a paste, the composition comprising: at least one thickener in an amount of about 0.1 wt% to about 8 wt%, at least one organic solvent and humectant in an amount of about 20 wt% to about 70 wt%, at least one emulsifier and rheology modifier in an amount of about 1.5 wt% to about 10 wt%, at least one emollient in an amount of about 2 wt% to about 35 wt%, and at least one conditioning agent in an amount of about 5 wt% to about 40 wt%; wherein the conditioner composition is free of, or substantially free of, water.

In an implementation, the at least one thickener is caesalpinia spinosa gum.

In yet another implementation, the at least one organic solvent and humectant is a polyol.

In yet another implementation, the at least one organic solvent and humectant is glycerin.

In yet another implementation, the at least one organic solvent and humectant is glycerin, or other diol, glycol, aloe vera gel, sodium hyaluronate (can act as an active), hydrolyzed hyaluronic acid (can act as an active), phenoxyethanol (can be used as a preservative), and/or lactic acid (can also be used as a pH adjuster).

In yet another implementation, the at least one emulsifier and rheology modifier is jojoba esters.

In yet another implementation, the at least one emulsifier and rheology modifier is jojoba esters, Polyglyceryl-3 Stearate, Cetearyl Alcohol, PCA Glyceryl Oleate, Glyceryl Caprylate, Brassicyl Isoleucinate esylate, and/or Brassica Alcohol.

In yet another implementation, the at least one emollient is glyceryl caprylate/caprate.

In yet another implementation, the at least one conditioning agent is Arachidyl/Behenyl Betainate Esylate and Arachidyl/Behenyl Alcohol.

In yet another implementation, the at least one conditioning agent is Arachidyl/Behenyl Betainate Esylate, Arachidyl/Behenyl Alcohol, PCA glyceryl oleate, Polyglyceryl-3 Betainate Acetate, Lauryl/Myristyl Polyricinoleate, and/or Glycerin.

In yet another implementation, the composition further comprises at least one surfactant, for example, sodium cocoyl isothionate.

In yet another implementation, the composition further comprises at least one active, for example, algae oil, essential oil blend, and/or hyaluronic acid.

In yet another implementation, the composition further comprises dead sea salt coarse.

In yet another implementation, the composition is used as a conditioner or a hair mask.

### DETAILED DESCRIPTION

It is an object of the following to provide concentrated cosmetic compositions for shampooing, cleansing, washing, moisturizing, conditioning, and/or exfoliating the hair and skin. More particularly, an object of the present disclosure is to provide hair care and body care pastes that are substantially or completely waterless or have no added water. With the recent focus on more sustainable living choices and use of natural products, in a preferred embodiment, the cosmetic compositions are comprised of ingredients of which a substantial majority or all of the ingredients are plant-derived, biodegradable, vegan, and environmentally friendly. Furthermore, it is preferred if all of the ingredients are cruelty-fee, sulfate-free, paraben-free, silicone-free, synthetic fragrance-free and does not contain added synthetic preservatives. Clean ingredients are not only important for what goes on our skin, but also what goes down our shower drains and into our water systems. Such restrictions clearly add challenges when formulating a product that has sufficient lather, an acceptable texture, a pleasing scent, a pleasing colour, and sufficient performance (cleaning and/or conditioning). The waterless nature of the product is advantageous in that there is reduced shipping cost, reduced carbon footprint, reduced volume, and reduced packaging waste when compared with traditional liquid products. In the preferred embodiments, the compositions are a thick paste, paste-like or semi-solid viscosity. This consistency allows for the compositions to be packaged in recyclable tubes without the use of single-use plastics. In the preferred embodiment, the tubes are made of recyclable aluminum. The paste-like consistency has the further advantage of allowing the user to spread the composition to facilitate easy dissolving and lather when mixed with water. This paste-to-liquid transformation is quick and much closer to what users are used to with liquid products, making it an easy eco-upgrade for wider adoption.

The term “hair care” as used herein will be understood to refer to a shampoo, hair wash, conditioner, or hair mask composition, or any combination thereof. For example, a shampoo composition may be understood to be conditioning to some extent; a shampoo composition may be understood to perform additionally as a body wash composition; and/or a conditioner composition may be understood to perform additionally as a hair mask composition. The term “body care” as used herein will be understood to refer to a body wash, face wash, hand wash, or face cleanser composition, or any combination thereof. For example, a body wash composition may be understood to perform additionally as a face cleanser. The term “anhydrous paste” as used herein will be understood to refer to a composition in the form of mostly, substantially, or completely waterless paste which comprises at least one organic solvent. The terms “cosmetic paste” and “cosmetic concentrate paste” as used herein will be understood to refer to hair care or body care concentrates in the form of an anhydrous or waterless paste. For the purposes of this disclosure, “waterless” is defined as no added water. In the preferred embodiment, there is less than 10 percent water. In one embodiment, there is between 6% and 10% water. In the preferred embodiment, there is less than 6% percent water.

The disclosure relates to a cosmetic concentrate paste which, unlike solid hair care and body care compositions such as dry shampoo, solid shampoo or conditioner bars, powders, or tabs, the paste may be conveniently dispensed from a tube or bottle, such as an aluminum tube, in a small, easily repeatable amount (e.g., a half-inch to one-inch strip). Additionally, in contrast to known hair care or body care concentrates, such cosmetic paste may be free of, or substantially free of, water, which in turn may improve its resistance to bacterial contamination, thereby enabling the use of little to no traditional preservatives. The term “substantially free of” as used herein will be understood to refer to about 10 wt% of less, 6 wt% or less, about 3 wt% or less, or about 1 wt% or less of a stated ingredient. The term “free of” as used herein will be understood to refer to negligible, incidental, or, no detectable amount of the stated ingredient or thing. The cosmetic paste may be quickly transformed into a creamy liquid when water is added (for example, in the shower) which, unlike bars and powders, may result in a sensorial experience similar to what users are already used to with liquid hair care and body care products. The cosmetic paste therefore allows for easier adoption by the user, with additional eco-benefits of the concentrated format such as lower carbon emissions from shipping and less packaging waste.

As discussed in greater detail below, a number of challenges were encountered in creating waterless cosmetic pastes that contain little or no preservatives and substantially or exclusively environmentally friendly components, while having satisfactory cosmetic and aesthetic properties. The inventors encountered substantial difficulty dissolving in a non-aqueous solvent a quantity of environmentally friendly surfactants sufficient to achieve acceptable cleaning performance. The inventors also encountered difficulty in creating a substantially waterless conditioner, since conditioners are typically oil in water emulsions.

Such cosmetic properties may include, for example, performance in cleaning /moisturizing hair/ skin, rinsing off easily, causing hair to be soft to the touch, creating satisfactory foaming/lather when in contact with water, causing hair to appear shiny, causing hair to feel voluminous and lightweight, causing hair to have “slip” during application, causing detangling or easy wet and dry combing and leaving a soft silky feel after being washed off. Such aesthetic properties may include, for example, having a desirable color such as a white or cream color, thermal stability, having a desirable texture such as smooth and free from particulates, and having a desirable smell.

The inventors have found that combining particular amounts of at least one surfactant with at least one organic solvent, at least one emollient, and at least one thickener can result in a shampoo paste having sufficiently satisfactory cosmetic properties, particularly surprisingly effective cleaning. The inventors have also found that combining particular amounts of at least one thickener with at least one organic solvent and humectant, at least one emulsifier and rheology modifier, at least one emollient, and at least one conditioning agent can result in a composition having sufficiently satisfactory cosmetic properties.

The cosmetic paste of the present disclosure may include thickeners with good water solubility requiring low or minimal shear stress, such as from rubbing hands, to thicken the waterless paste when activated by water on application to achieve desired lather or suds or cream as found in traditional liquid-based products.

The shampoo paste may comprise at least one surfactant at a concentration of about 15 wt% to about 48 wt%, at least one thickener at a concentration of about 0 wt% to about 10 wt%, at least one emollient at a concentration of about 0 wt% to about 15 wt%, and at least one organic solvent at a concentration of about 25 wt% to about 60 wt%. The at least one surfactant may be present in an amount of, for example, about 30 wt% to about 40 wt%. The at least one thickener may be present in an amount of, for example, about 0.5 wt% to about 2.5 wt%. The at least one emollient may be present in an amount of, for example, about 1 wt% to about 5 wt%. The at least one organic solvent may be present in an amount of, for example, about 55 wt% to about 60 wt%. In one embodiment, the shampoo paste may comprise a conditioning agent in the range of about 0 wt% to 30 wt%.

Preferably, the shampoo is free of, or substantially free of, water. For example, the shampoo may have a water content of 10% or less, 6% or less, 3% or less, or 1% or less. It can be appreciated that the shampoo may have a water content of above 0 wt% depending on, e.g., the reagents used and their incidental water contents. The shampoo may have a pH of, for example, about 5 to about 5.7, from about 5.6 to about 7.0, or from about 6.2 to about 7.7. The shampoo may have a viscosity of about 5,000 cps to about 85,000 cps at 40° C. in production and may appear as a highly viscous paste after 24 hours at room temperature. In a preferred embodiment, the shampoo may have a viscosity of about 15,000 cps to about 65,000 cps at 40° C.

In a preferred embodiment, the at least one surfactant may include a primary surfactant and a secondary surfactant, and the primary surfactant may be, for example, sodium lauroyl glutamate and the secondary surfactant may be, for example, sodium cocoyl isothionate. In a preferred embodiment, the at least one thickener may be, for example, lysolecithin, sclerotium gum, xanthan gum, and/or pullulan. In a preferred embodiment, the at least one emollient may be, for example, glyceryl caprylate/caprate. In a preferred embodiment, the at least one organic solvent may be, for example, glycerin and may further comprise propandiol and/or aloe vera gel. In a preferred embodiment, the shampoo paste may further comprise an oil such as, for example, Amla oil which may encourage shine and a generally healthy appearance of hair.

The shampoo paste may further comprise additives such as, for example, essential oil blends, one or more suitable chelating agents and one or more suitable pH adjusters. Suitable chelating agents may include, but are not limited to, trisodium ethylenediamine disuccinate and sodium citrate. Suitable pH adjusters may include, but are not limited to, sodium bicarbonate, sodium hydroxide and L-arginine.

The at least one surfactant may be anionic, amphoteric, or non-ionic. Preferably, the at least one surfactant is substantially or completely water-free. Preferably, the at least one surfactant is an anionic surfactant. Even more preferably, the at least one surfactant is a mild and/or gentle surfactant. Even more preferably, the at least one surfactant is plant derived. Even more preferably, the at least one surfactant is amino acid based. The at least one surfactant may be, for example, Disodium Cocoyl Glutamate, Disodium Laureth Sulfosuccinate, Potassium Cocoate, Potassium Cocoyl Glutamate, Potassium Cocoyl Glycinate, Potassium Lauroyl Glutamate, Potassium Lauroyl Sarcosinate, Potassium Myristoyl Glutamate, Sodium Cocoyl Alaninate, Sodium Cocoyl Glutamate, Sodium Cocoyl Glycinate, Sodium Cocoyl Isethionate, Sodium Cocoyl Methyl Isethionate, Sodium Cocoyl Sarcosinate, Sodium Cocoyl Threoninate, Sodium Lauroyl Glutamate, Sodium Lauroyl Glycinate, Sodium Lauroyl Isethionate, Sodium Lauroyl Lactylate, Sodium Lauroyl Methyl Isethionate, Sodium Lauroyl Sarcosinate, Sodium Methyl Cocoyl Taurate, Sodium Methyl Lauroyl Taurate, Sodium Myristoyl Glutamate, Sodium Myristoyl Sarcosinate, Sodium Stearoyl Glutamate, Sucrose Palmitate, Sucrose Stearate, TEA-Cocoyl Alaninate, TEA-Cocoyl Glutamate, TEA-Lauroyl Glutamate, TEA-Lauroyl Sarcosinate, or a combination thereof. The at least one surfactant may include one or more other suitable surfactants. The at least one surfactant may be in solid form, including but not limited to a powder or pellet.

The at least one thickener may also be an emulsifier or a stabilizer. The at least one thickener may be one or more of, for example, a cationic or quaternary ammonium derivative of guar gum. The at least one thickener may have good water solubility requiring low or minimal shear stress, such as from rubbing hands, to thicken the waterless paste when activated by water on application to achieve desired lather or suds or cream as found in traditional liquid-based products. Preferably, the at least one thickener is substantially or completely water free. Even more preferably, the at least one thickener is plant derived. The at least one thickener may be, for example, Acacia Senegal Gum, Algin, Beta-Glucan, Betula Alba Juice, Butylene Glycol, Caesalpinia Spinosa Gum, Caprylic/Capric Triglyceride, Cellulose, Cellulose Gum, Ceratonia Siliqua (Carob) Gum, Chondrus Crispus Extract, Chondrus Crispus Powder, Chlorphenesin, Citric Acid, Cyamopsis Tetragonoloba (Guar) Gum, Glucose, Glycerin, Glyceryl Caprylate/Caprate, Guar Hydroxypropyltrimonium Chloride, Hydroxyethylcellulose, Hydroxypropyl Methylcellulose, Hydroxypropyl Starch Phosphate, Lecithin, Lysolecithin, Microcrystalline Cellulose, Natto Gum, Phenoxyethanol, Polyurethane-79, Pullulan, Sclerotium Gum, tapioca starch, Xanthan Gum, or a combination thereof. The at least one thickener may include one or more other suitable thickeners.

Preferably, the at least one emollient is plant derived. The at least one emollient may be, for example, Amla oil, C8-C18 Alkyl Ester, Caprylic/Capric Triglyceride, Coco-Caprylate, Coco-Caprylate/Caprate, Coconut Alkane, coconut oil, Dimer Dilinoleate, Dimer Dilinoleyl, Glyceryl Abietate, Glyceryl Caprylate/Caprate, Glyceryl Isostearate, Glyceryl Laurate, Glyceryl Linoleate, Glyceryl Linolenate, Glyceryl Oleate, Hydrogenated Olive Oil Unsaponifiables, Isoamyl Caprate-Caprylate, Isononyl Isononanoate, MCT oil, Propanediol Dicaprylate/Caprate, Propylene Glycol Dicaprate, shea butter, or a combination thereof. The at least one emollient may include one or more other suitable emollients.

Preferably, the at least one organic solvent is substantially or completely water-free. Even more preferably, the at least one organic solvent is plant derived. Even more preferably, the at least one organic solvent is capable of solubilizing completely or substantially water-free ingredients. The at least one organic solvent may be, for example, 1,2-Hexanediol, 1,5-Pentanediol, aloe vera, aloe vera extract, Butylene Glycol, Dipropylene Glycol, flower water, fruit juice, glycerin, Hexylene Glycol, Methylpropanediol, Pentylene Glycol, Phenoxyisopropanol, Phenoxypropanediol, plant extracts, Propylene Glycol, Sorbitol, or a combination thereof. The at least one organic solvent may alternatively or in combination include one or more other suitable organic solvents.

Preferably, the body wash paste compositions or shampoo paste compositions will additionally include one or more conditioning agents. Even more preferably, the at least one conditioning agent is substantially or completely water-free. Even more preferably, the at least one conditioning agent is plant derived. The at least one conditioning agent may be, for example, Arachidyl/Behenyl Alcohol, Arachidyl/Behenyl Betainate Esylate, Behenamidopropyl, Dimethylamine, Behentrimonium Chloride, Behentrimonium Methosulfate, Behenyl Alcohol, Bis-(Ethyl PPG-3 Behenate) Dimonium Methosulfate, Bis-(Isostearoyl/Oleoyl Isopropyl), Dimonium Methosulfate, Brassica Alcohol, Brassicamidopropyl Dimethylamine, Brassicyl Valinate Esylate, Butylene Glycol, Cetearyl Alcohol (can also act as a thickener), Cetrimonium Chloride, Cetyl Alcohol (can also act as a thickener), Cocamidopropyl PG-Dimonium Chloride, Dioleoylethyl Hydroxyethylmonium Methosulfate, Dipalmitoylethyl Hydroxyethylmonium Methosulfate, Distearoylethyl Dimonium Chloride, Distearyldimonium Chloride, Disunfloweroylethyl Dimonium Chloride, fatty acids, Glycerin, Glyceryl Caprylate, Guar Hydroxypropyltrimonium Chloride, Hydrolyzed Rice Protein, Hydroxyethyl Cetearamidopropyldimonium Chloride, Lauryl Lactyl Lactate, Lauryl/Myristyl Polyricinoleate, Maranta Arundinacea root powder derivates, Myristyl Lactate, Oryza Sativa starch derivatives, PCA Glyceryl Oleate, Polyglyceryl-3 Betainate Acetate (can also act as an active), Polyglyceryl-3 Stearate, Polyquaternium-116, Propylene Glycol, Quaternium-98, Stearalkonium Chloride, Stearyl Alcohol, Sunflower Seed Oil Glycerides, Theobroma Cacao Seed Butter, or a combination thereof. The at least one conditioning agent may alternatively or in combination include one or more other suitable organic solvents.

It will be understood by those of ordinary skill in the art that the shampoo pastes ingredients described herein may be classified under more than one ingredient category depending on their weight percentage in the formula and intended function. For example, certain conditioning agents may additionally or alternatively act as surfactants when used in lower weight percentages. Such instances will be understood not to depart from the spirit and scope of the categories as disclosed herein.

Preferably, the ingredients comprising the shampoo paste are plant-based, biodegradable, vegan, and cruelty-free. Even more preferably, the shampoo paste composition is free from silicone-based and petroleum-derived ingredients. Even more preferably, the shampoo paste composition is free from added synthetic preservatives, such as parabens.

The final quality and specification of the shampoo paste may be significantly procedure dependent. The final quality of the product may be determined by physical, cosmetic, and aesthetic properties, including but not limited to texture of the paste, viscosity of the paste, foamability/lather, and stability of the paste. The important procedural factors may be, for example, order of addition of the ingredients, mixing speed, and temperature. In particular, the order of addition of the ingredients may be impacted by the dissolution compatibility between the at least one surfactant and the at least one organic solvent, and the physical form of the at least one surfactant.

A number of challenges were overcome in order to achieve waterless shampoo pastes that were free of, or substantially free of, water, and consisting of substantially or exclusively plant-derived components, while having satisfactory cosmetic and aesthetic properties. In particular, there is substantial difficulty dissolving in a non-aqueous solvent, a quantity of environmentally friendly surfactants sufficient to achieve acceptable cleaning performance. For example, in the preferred embodiment illustrated in Examples 1A-D, the shampoo paste comprised an optimal concentration of solid surfactants to achieve high foamability and lather without causing excessive hardness of the final product. These surfactants are assumed by manufacturers to be dissolved in water. To overcome challenges associated with dissolution of surfactants in water-free solvents, relatively high concentrations of such organic solvents were necessary. This caused the additional challenge of balancing surfactant solubility with the desired paste texture. In particular, it can be appreciated that anionic, plant-based, and water-free surfactants dissolve more efficiently in aloe vera gel than glycerin, but high concentrations of aloe vera gel (i.e., above 10 wt%) result in an unsatisfactorily sticky paste texture. Furthermore, it was determined that the physical form of the surfactants is critical to the dissolution process. In particular, reducing the granularity of the surfactant, (i.e., using powder rather than pellet surfactants) increases the total surface area of the solute and therefore improves the dissolution process.

Achieving an optimal viscosity using waterless or anhydrous ingredients to satisfy rheological requirements presents a further challenge. In order to circumvent the poor administration efficacy of shampoo bars and powders, as well as overcome the likelihood of wasting excess product associated with liquid shampoos and the carbon footprint and packaging waste they leave behind, the inventors of the present disclosure developed a semi-solid delivery method. To achieve convenient dispensation in a small, easily repeatable amount, this semi-solid paste may then be delivered to the user through a rigid or semi-rigid container, such as an aluminum tube. Eliminating water from the formula resulted in a significantly hard product that was not compatible with this delivery mechanism. To overcome this challenge, a low-viscosity solvent such as propanediol was included in the composition.

Furthermore, achieving heat-dependent property stability of the formula is yet a further challenge faced in the industry of natural cosmetic products. For example, silicone replacement esters, while beneficial in terms of decreasing the resistance of the final product and therefore improving rheological compatibility, could not be added to the composition as they cause separation. To overcome this challenge, lipid-based actives such as amla oil may be used instead.

In one embodiment, the shampoo composition may be adapted to perform as a body wash composition. It can be appreciated that the body wash composition may require a less viscous, softer texture in order to satisfy traditional sensorial expectations while maintaining lathering property and stability. To overcome a tradeoff between stability, viscosity, lather, and texture, it can be appreciated that sea salt may be added to stabilize the formulation. It will also be appreciated that the body wash composition may require the addition of conditioning agents, or the varying concentrations of conditioning agents. In one embodiment, Polyglyceryl-3 Betainate Acetate is the preferred conditioning agent.

The shampoo paste according to the present disclosure may be prepared, for example, by the following procedure. 1. Dissolve each surfactant separately in a mixture of the at least one solvent at approximately 45-55° C., wherein one part surfactant is dissolved in at least one part solvent. 2. After dissolution, mix surfactants until uniform. 3. Combine the solvent and the at least one thickener at approximately 45-55° C., then add to the mixture from step 2 and mix until uniform. 4. Combine the at least one emollient and any additives or oils, then add to the mixture from step 2 and mix until uniform while maintaining approximately 45-55° C. 5. Remove the mixture at 45-55° C. and allow the mixture to cool to room temperature. An ordinary person skilled in the art will recognize that the above procedure may be modified without departing from the spirit and scope disclosed herein to accommodate the physical and chemical differences among ingredients. For example, the mixture may be allowed to cool below 45° C. before the addition of additives.

The shampoo paste may have a viscosity of 5,000 to 85,000 cps at 40° C. in process before being cooled to room temperature, in order to achieve rheological compatibility with the delivery mechanism. In a preferred embodiment, the shampoo may have a viscosity of about 15,000 cps to about 65,000 cps at 40° C. The shampoo may have a white or cream colour and a smooth, homogenous texture free from particulates or clumps. The shampoo paste can be expected to activate upon the addition of water and shear stress, such as when rubbing the shampoo paste between wet hands and subsequently lathering the shampoo paste in wet hair or on wet skin, resulting in an expansion of the composition and significant foaming.

The conditioner paste may comprise at least one thickener in an amount of about 0.1 wt% to about 8 wt%, at least one organic solvent and humectant in an amount of about 5 wt% to about 70 wt%, at least one emulsifier and rheology modifier in an amount of about 1.5 wt% to about 10 wt%, at least one emollient in an amount of about 2 wt% to about 35 wt%, and at least one conditioning agent in an amount of about 5 wt% to about 60 wt%. The at least one thickener may be present in an amount of, for example, about 0.5 wt% to about 1.5 wt%. The at least one organic solvent and humectant may be present in an amount of, for example, about 55 wt% to about 65 wt%. The at least one emulsifier and rheology modifier may be present in an amount of, for example, 3 wt% to about 6 wt%. The at least one emollient may be present in an amount of, for example, 3 wt% to about 7 wt%. The at least one conditioning agent may be present in an amount of, for example, about 12 wt% to about 30 wt%. It will be understood by those of ordinary skill in the art that the ranges described herein are instructional and may be deviated from without departing from the spirit and scope of the invention as disclosed herein. In a preferred embodiment, the conditioner paste may be a hair mask. In this embodiment, the conditioner paste composition can further include an active ingredient, and/or an antioxidant. For example, the active ingredient, and/or an antioxidant can include a tocopherol (Vitamin E), Vitamins such as F and C, Phospholipids, Ceramides, Amino acids and proteins, and Natural sugars derivatives.

Preferably, the conditioner paste is free of, or substantially free of, water. For the purposes of the conditioner in this disclosure, substantially free of water is defined as the conditioner paste having a water content of 10% or less. In a preferred embodiment, substantially free of water is interpreted as 6% or less. In another preferred embodiment, substantially free of water is interpreted as 3% or less. In yet a further preferred embodiment, substantially free of water is 1% or less. In yet another embodiment, there is no added water in the conditioner. The advantage of decreased water content lies in decreased weight and volume of the product when compared to traditional liquid or cream products. This leads to decreased transport costs, lower carbon footprint, less packaging waste and other environmental benefits including reduced water consumption in manufacturing, while instead using the water you already use in the shower. It can be appreciated that the conditioner paste may have a water content of above 0 wt% depending on, e.g., the reagents used and their incidental water contents. The conditioner paste may have a pH of, for example, from about 3.0 to about 6.5. The conditioner paste may have a viscosity of, for example, about 50,000 cps to about 95,000 cps at 40° C. in production and may appear as a highly viscous paste after 24 hours at room temperature. In a preferred embodiment, the conditioner composition may have a viscosity of about 50,000 cps to about 80,000 cps at 40° C.

In a preferred example embodiment, the least one thickener may be, for example, caesalpinia spinosa gum. In a preferred example embodiment, the at least one organic solvent and humectant may be, for example, glycerin or other diol, glycol and may further comprise aloe vera gel, sodium hyaluronate, and hydrolyzed hyaluronic acid. In a preferred embodiment, the at least one emulsifier and rheology modifier may be, for example, jojoba esters, and may further comprise Polyglyceryl-3 Stearate, Cetearyl Alcohol, PCA Glyceryl Oleate, Glyceryl Caprylate, Brassicyl Isoleucinate esylate, Brassica Alcohol, or a combination thereof. In a preferred embodiment, the at least one emollient may be, for example, glyceryl caprylate/caprate. In a preferred example embodiment, the at least one conditioning agent may be, for example, PCA Glyceryl Oleate, Polyglyceryl-3 Betainate Acetate, Arachidyl/Behenyl Betainate Esylate, Arachidyl/Behenyl Alcohol, Lauryl/Myristyl Polyricinoleate, Glycerin, or a combination thereof. In a preferred embodiment, the conditioner paste may further comprise at least one surfactant, for example, sodium cocoyl isethionate, and/or at least one active, for example, algae oil, or Triolein.

The conditioner paste may further comprise additives such as, for example, essential oil blends, one or more suitable chelating agents and one or more suitable pH adjusters. Suitable chelating agents may include, but are not limited to, trisodium ethylenediamine disuccinate and sodium citrate. Suitable pH adjusters may include, but are not limited to, sodium bicarbonate, sodium hydroxide and L-arginine.

The at least one thickener may be one or more of, for example, a cationic or quaternary ammonium derivative of guar gum. The at least one thickener may have good water solubility requiring low or minimal shear stress, such as from rubbing hands, to transform the waterless paste when activated by water on application to achieve a desired creamy liquid as found in traditional liquid-based products. Preferably, the at least one thickener is plant derived. The at least one thickener may be, for example, Acacia Senegal Gum, Algin, Beta-Glucan, Betula Alba Juice, Butylene Glycol, Caesalpinia Spinosa Gum, Cellulose, Cellulose Gum, Ceratonia Siliqua (Carob) Gum, Chondrus Crispus Extract, Chondrus Crispus Powder, Cyamopsis Tetragonoloba (Guar) Gum, Glucose, Glycerin, Guar Hydroxypropyltrimonium Chloride, Hydroxyethylcellulose, Hydroxypropyl Methylcellulose, Hydroxypropyl Starch Phosphate, Lecithin, Lysolecithin, Microcrystalline Cellulose, Natto Gum, Polyurethane-79, Pullulan, Sclerotium Gum, Sodium Carboxymethyl Starch, tapioca starch, Xanthan Gum, Hydroxypropyltrimonium Inulin, or a combination thereof. The at least one thickener may alternatively or in combination include one or more other suitable thickeners.

Preferably, the at least one organic solvent and humectant is substantially or completely water-free. Even more preferably, the at least one organic solvent and humectant is plant derived. Even more preferably, the at least one organic solvent and humectant is capable of solubilizing completely or substantially water-free ingredients. The at least one organic solvent and humectant may be, for example, 1,2-Hexanediol, 1,5-Pentanediol, aloe vera, aloe vera extract, Butylene Glycol, castor oil, Dipropylene Glycol, flower water, fruit juice, glycerin, Hexylene Glycol, Hydrolyzed Hyaluronic Acid, Pentylene Glycol, Phenoxypropanediol, plant extracts, Propylene Glycol, Sodium Hyaluronate, Sorbitol, or a combination thereof. The at least one organic solvent and humectant may alternatively or in combination include one or more other suitable organic solvents.

Preferably, the at least one emulsifier and rheology modifier is plant derived. Even more preferably, the at least one emulsifier and rheology modifier is a quaternary surfactant(“quat”)-free and polyethylene glycol (“PEG”)-free emulsifier with cationic charge. The at least one emulsifier and rheology modifier may be, for example, Brassica Alcohol, Brassicyl Isoleucinate esylate, Cetearyl Alcohol, Glyceryl Caprylate, Jojoba esters, Polyglyceryl-3 Stearate, PCA Glyceryl Oleate, or a combination thereof. The at least one emulsifier may include one or more other suitable emulsifiers. The emulsifier may be a water in oil (W/O) emulsifier. In one embodiment, the W/O emulsifier may be, for example, Polyglyceryl-4 Olivate/Polyricinoleate.

Preferably, the at least one emollient is plant derived. The at least one emollient may be, for example, Amla oil, Simmondsia Chinensis (Jojoba) Seed Oil, C8-C18 Alkyl Ester, Caprylic/Capric Triglyceride, Coco-Caprylate, Coco-Caprylate/Caprate, Coconut Alkane, coconut oil, Dimer Dilinoleate, Dimer Dilinoleyl, Glyceryl Abietate, Glyceryl Caprylate/Caprate, Glyceryl Isostearate, Glyceryl Laurate, Glyceryl Linoleate, Glyceryl Linolenate, Glyceryl Oleate, Hydrogenated Olive Oil Unsaponifiables, Isoamyl Caprate-Caprylate, Isononyl Isononanoate, MCT oil, Propanediol Dicaprylate/Caprate, Propylene Glycol Dicaprate, shea butter, or a combination thereof. The at least one emollient may alternatively or in combination include one or more other suitable emollients.

The at least one conditioning agent may be a cationic active. Preferably, the at least one conditioning agent is plant derived. The at least one conditioning agent may be, for example, Arachidyl/Behenyl Alcohol, Arachidyl/Behenyl Betainate Esylate, Behenamidopropyl, Dimethylamine, Behentrimonium Chloride, Behentrimonium Methosulfate, Behenyl Alcohol, Bis-(Ethyl PPG-3 Behenate) Dimonium Methosulfate, Bis-(Isostearoyl/Oleoyl Isopropyl), Dimonium Methosulfate, Brassica Alcohol, Brassicamidopropyl Dimethylamine, Brassicyl Valinate Esylate, Butylene Glycol, Cetearyl Alcohol, Cetrimonium Chloride, Cetyl Alcohol, Cocamidopropyl PG-Dimonium Chloride, Dioleoylethyl Hydroxyethylmonium Methosulfate, Dipalmitoylethyl Hydroxyethylmonium Methosulfate, Distearoylethyl Dimonium Chloride, Distearyldimonium Chloride, Disunfloweroylethyl Dimonium Chloride, fatty acids, Glycerin, Guar Hydroxypropyltrimonium Chloride, Hydrolyzed Rice Protein, Hydroxyethyl Cetearamidopropyldimonium Chloride, Lauryl Lactyl Lactate, Lauryl/Myristyl Polyricinoleate, Maranta Arundinacea root powder derivates, Myristyl Lactate, Oryza Sativa starch derivatives, PCA Glyceryl Oleate, Polyglyceryl-3 Betainate Acetate, Polyquaternium-116, Propylene Glycol, Quaternium-98, Shea Butter, Stearalkonium Chloride, Stearyl Alcohol, Sunflower Seed Oil Glycerides, Theobroma Cacao Seed Butter, Crambe Abyssinica Seed Oil, Phytosterols, Oleyl Alcohol, or a combination thereof. The at least one conditioning agent may include one or more other suitable conditioning agents.

In a preferred embodiment, the conditioner paste may further comprise at least one surfactant in an amount of about 0.3 wt% to about 15 wt%. The at least one surfactant may be present in an amount of, for example, about 3 wt% to about 7 wt%. The at least one surfactant may be an anionic surfactant, cationic surfactant, non-ionic surfactant, amphoteric surfactant, or a combination thereof. Preferably, the at least one surfactant is an anionic surfactant. Even more preferably, the at least one surfactant is a mild and/or gentle surfactant. Even more preferably, the at least one surfactant is plant derived. Even more preferably, the at least one surfactant is amino acid based. In a preferred example embodiment, the at least one surfactant is a secondary surfactant which may be, for example, sodium cocoyl isothionate. In combination or in the alternative, the at least one surfactant may be, for example, Disodium Cocoyl Glutamate, Disodium Laureth Sulfosuccinate, Potassium Cocoate, Potassium Cocoyl Glutamate, Potassium Cocoyl Glycinate, Potassium Lauroyl Glutamate, Potassium Lauroyl Sarcosinate, Potassium Myristoyl Glutamate, Sodium Cocoyl Alaninate, Sodium Cocoyl Glutamate, Sodium Cocoyl Glycinate, Sodium Cocoyl Isethionate, Sodium Cocoyl Methyl Isethionate, Sodium Cocoyl Sarcosinate, Sodium Cocoyl Threoninate, Sodium Lauroyl Glutamate, Sodium Lauroyl Glycinate, Sodium Lauroyl Isethionate, Sodium Lauroyl Lactylate, Sodium Lauroyl Methyl Isethionate, Sodium Lauroyl Sarcosinate, Sodium Methyl Cocoyl Taurate, Sodium Methyl Lauroyl Taurate, Sodium Myristoyl Glutamate, Sodium Myristoyl Sarcosinate, Sodium Stearoyl Glutamate, Sucrose Palmitate, Sucrose Stearate, TEA-Cocoyl Alaninate, TEA-Cocoyl Glutamate, TEA-Lauroyl Glutamate, TEA-Lauroyl Sarcosinate, or a combination thereof. The at least one surfactant may include one or more other suitable surfactants.

In a preferred embodiment, the conditioner paste may further comprise at least one active in an amount of about 1 wt% to about 10 wt%. The at least one active may be present in an amount of, for example, about 1 wt% to about 4 wt%. Preferably, the at least one active is plant derived. In a preferred example embodiment, the at least one active may be, for example, algae oil, Ricinus Communis (Castor) Seed Oil, Camellia Japonica Seed Oil, Camellia Sinensis Seed Oil, Camellia Japonica Flower Extract, Camellia Japonica Seed Extract, Crambe Abyssinica Seed Oil, Phytosterols, Oleyl Alcohol, or a combination thereof. In combination or in the alternative, the at least one active may be, for example, algae oil, Triolein, Amla oil, coconut oil, essential oil blend, hylauronic acid, Hydrolyzed Hyaluronic acid, phospholipids, ceramides, MCT oil, plant and seed oils, plant extracts, shea butter, or a combination thereof.

It will be understood by those of ordinary skill in the art that the conditioner paste ingredients described herein may be classified under more than one ingredient category depending on their weight percentage in the formula and intended function. For example, certain conditioning agents may additionally or alternatively act as surfactants when used in lower weight percentages. Such instances will be understood not to depart from the spirit and scope of the categories as disclosed herein.

Preferably, the ingredients comprising the conditioner paste are plant-based, biodegradable, vegan, and cruelty-free. Even more preferably, the conditioner paste composition is free from silicone-based and petroleum-derived ingredients. Even more preferably, the conditioner paste composition is free from added synthetic preservatives such as parabens.

It may be that a hair mask paste may be formulated based on the conditioner paste according to the present disclosure. In a preferred example embodiment, the hair mask composition may comprise the at least one thickener in an amount of about 0.1 wt% to about 18 wt%, the at least one organic solvent and humectant in an amount of about 15 wt% to about 70 wt%, the at least one emulsifier and rheology modifier in an amount of about 1.5 wt% to about 40 wt%, the at least one emollient in an amount of about 2 wt% to about 70 wt%, and the at least one conditioning agent in an amount of about 5 wt% to about 60 wt%. The preferred example embodiment of the hair mask composition may additionally comprise the at least one surfactant in an amount of about 0.3 wt% to about 15 wt%, and/or the at least one active in an amount of about 1 wt% to about 10 wt%.

It will be understood by those of ordinary skill in the art that the hair mask paste ingredients described herein may be classified under more than one ingredient category depending on their weight percentage in the formula and intended function. For example, certain conditioning agents may additionally or alternatively act as surfactants when used in lower weight percentages. As an additional example, certain emollients may additionally or alternatively act as solvents when used in greater weight percentages. In such instances, the total amount of ingredients in the affected categories may appear to increase above or decrease below the disclosed ranges, but such instances will be understood not to depart from the spirit and scope of the categories as disclosed herein.

The final quality and specification of the conditioner paste may be significantly procedure dependent. The final quality of the product may be determined by physical, cosmetic, and aesthetic properties, including but not limited to texture of the paste, viscosity of the paste, stability of the paste, color of the paste, and conditioning and/or hydrating properties. The important procedural factors may be, for example, order of addition of the ingredients, mixing speed, mixing duration, and temperature. In particular, the mixing temperature and the order of addition of the ingredients may be impacted by the heat-dependent stability of various ingredients. For example, the miscibility and suspension of the oil phase in the solvent phase may be improved by adding the solvent to the oil rather than the reverse, and by combining all anhydrous ingredients prior to phase mixing to increase homogeneity.

A number of challenges were overcome in order to achieve high viscosity, waterless conditioner pastes that were free of, or substantially free of, water, and consisting of substantially or exclusively plant-derived components, while having satisfactory cosmetic and aesthetic properties. In particular, the inventors encountered substantial difficulty overcoming challenges associated with excessive adhesion caused by waxy and occlusive ingredients, heat-dependent instability, and conditioning performance of naturally derived ingredients.

Hair conditioning is achieved by lowering the friction between strands of hair through deposition of silicones and lipidic agents, or through electrostatic interaction with the positive charge of certain polymers and the negative charge on hair strands. A surface coating of silicones, lipidic agents, or cationic surfactants reduces resistance to movement and provides the hair with the desired manageability and sensory effects described as smoothness, softness, and shine. However, these desirable effects are often correlated with excessive tackiness associated with anhydrous materials which undermines lubricity and spreading or slip during application and creates a viscous film residue on the hair and scalp. A high oil/fat content is required to meet occlusion and lubricity requirements, but this normally results in an undesirable texture and excessive surface tension. Furthermore, a high oil/fat content directly correlates to decreased emulsion property and stability, necessitating the addition of thickeners and stabilizers to avoid oil separation.

The inventors faced a significant challenge in achieving efficient dispersion and miscibility of the functional ingredients in a waterless base while meeting the high viscosity and paste-like texture requirements, minimizing unwanted viscous film residue, and achieving the desired spreadability and slip during application. In particular, the use of a non-aqueous base, for example, glycerin, rather than the traditional water base, anhydrous combined with the desiccated state of the ingredients, significantly influenced the resulting active solubility as well as increased the textural density and viscosity of the formula, causing deficient spreadability and combability of the product during and after hair application. The high content of waxy molecules and fatty acids in the anhydrous actives led to undesired tackiness, oiliness, and adhesion. The desiccated state of the formula also resulted in compromised emulsion stability under thermal conditions.

To overcome the heavy, viscous film left on hair after rinsing due to the excessive adhesion caused by waxy lipid molecules and other occlusive ingredients, the inventors replaced these with anhydrous actives. A 100% anhydrous formula resulted in a poor texture, low viscosity, and oily performance. To achieve a satisfactory viscosity, the inventors reduced the percentage of anhydrous actives and increased the quantity of thickener. In addition, the inventors found that the addition of a surfactant, such as sodium cocoyl isethionate, improved miscibility with water during rinsing and reduced the viscous film.

An additional problem associated with the use of high percentages of anhydrous actives is that it causes difficulty in achieving stability due to the tendency of oils to separate at high temperature. This effect is exacerbated by the addition of natural silicone-like esters, such as isononyl isononanoate, which result in heat-induced phase separation and a decrease in conditioning effect and rheology. To overcome stability challenges, the inventors developed a combination of natural and biodegradable oils, at least one long chain polyester, and at least one cationic emulsifier, which resulted in surprisingly satisfactory sensory effects and manageability. Furthermore, the addition of a small percentage of salt improved stability.

Finally, to mimic the efficacy of traditional synthetic quaternary cationic agents using plant-derived ingredients, the inventors identified several naturally derived cationic agents with strong conditioning properties. These include algae oil, Arachidyl/Behenyl Betainate Esylate, Arachidyl/Behenyl Alcohol, Brassicyl Isoleucinate esylate (and) Brassica Alcohol, Polyglyceryl-3 Betainate Acetate, PCA glyceryl oleate, and Lauryl/Myristyl Polyricinoleate. Furthermore, the inventors found that reducing the pH of the system between 3-5 using citric acid improved the functionality of the conditioning agents.

The conditioner paste according to the present disclosure may be prepared, for example, by the following procedure. If applicable, the at least one surfactant is combined with a portion of the at least one solvent. The at least one thickener is combined with one portion of the at least one solvent. At least one active is combined with at least another portion of the solvent. The solvent mixtures from part 1 are combined, heated to approximately 60-80° C., and mixed until homogeneous. The at least one conditioning agents are combined, heated to at approximately 60-80° C., and mixed until melted. The at least one emollient and the at least one active are combined, then mixed into the mixture. The Solvent / glycol phase comprising of 3 mixtures is combined and heated to 60-80° C. then mixed with the emollient / oil phase mixture while homogenized. While homogenizing, the temperature is reduced. The mixture is then removed at 45-50° C. and allowed to cool to room temperature. A person skilled in the art will recognize that the above procedure may be modified without departing from the spirit and scope disclosed herein to accommodate the physical and chemical differences among ingredients.

The conditioner paste may have a preferred viscosity of 50,000 to 90,000 cps at 40° C. in process before being cooled to room temperature, in order to achieve rheological compatibility with the delivery mechanism. The conditioner paste may have a white or cream colour and a smooth, homogenous texture free from particulates or clumps. The conditioner paste can be expected to activate upon the addition of water and shear stress, such as when rubbing the conditioner paste between wet hands and subsequently massaging the conditioner paste in wet hair or on wet skin, resulting in transformation of the composition to a creamy liquid.

### EXAMPLES

The present disclosure is described and demonstrated in the following examples. The examples are given solely for the purpose of illustration and are not to be construed as limitations of the hair care and body care compositions presently disclosed as many variations thereof are possible without departing from the spirit and scope disclosed herein.

### Examples 1A-C: Shampoo Compositions

Different formulations of the present disclosure are presented herein as illustrations of various shampoo paste compositions. Three shampoo pastes were formulated with the following components in the amounts indicated.

| | Example 1A | Example 1B | Example 1C |
|---|---|---|---|
| Ingredient | W/W% | w/w% | w/w% |
| Sodium Cocoyl Isothionate | 15 | 15 | 15 |
| Sodium Lauryl Glutamate | 20 | | |
| Sodium Stearoyl Glutamate | | 20 | |
| Sodium Cocoyl Glycinate | | | 20 |
| Glycerin | 39.5 | 39.5 | 39.5 |
| Aloe Vera Gel | 17.8 | 17.8 | 17.8 |
| Propanediol | 2 | 2 | 2 |
| Lysolecithin (And) Sclerotium Gum (And) Xanthan Gum (And) Pullulan | 0.5 | | 0.5 |
| Xanthan Gum (And) Lecithin (And) Sclerotium Gum (And) Pullulan | | 0.5 | |
| Glyceryl Caprylate/Caprate | 2 | 2 | 2 |
| Amla Oil | 0.2 | 0.2 | 0.2 |
| Essential Oil Blend | 3 | 3 | 3 |
| TOTAL | 100 | 100 | 100 |
| Viscosity (at 40C) (cP) | 50,000-85,000 | n/a | 17,190-21,010 |
| Consistency at room temperature | Thick paste | thick paste | thick paste |
| pH | 5.50-6.8 | 5.65-6.91 | 6.24-7.62 |
| Specific gravity | | 0.86-0.99 | 0.7-0.86 |
| Solid wt% | 78.00-88.00 | | |
| Colour | White | White | White |
| Textu re | Smooth paste | Whipped paste | Thick sticky paste |
| Scent | Citrus | Citrus | Citrus |
| Foamability | High | Medium-high | High |
| Cleaning | High | Medium-high | High |

Preparation of Example 1A was as follows:

1. Dissolve the primary surfactant, in a mixture of approximately 14-25 wt% glycerin and approximately 9 wt% aloe vera at approximately 55° C.

2. Dissolve the secondary surfactant, in a mixture of 14-25 wt% glycerin and approximately 9 wt% aloe vera at approximately 55° C.

3. Combine the mixture from step 1 into the mixture from step 2 and continued to mix at approximately 55-65° C. until the mixture looks smooth and uniform.

4. Combine the propanediol and the thickener at approximately 55° C., then added it to the mixture from step 3 and mixed until uniform.

5. Combined the emollient, Amla oil, and essential oils at room temperature, then added it to the mixture from step 3 and mixed until uniform while maintaining approximately 55° C.

6. Allowed the mixture to cool to room temperature.

The shampoo composition according to Example 1A had a homogenous and paste-like texture that was easily removed from the application tube and malleable in the user’s hands. The shampoo composition activated upon addition of water and resulted in a rich, creamy lather with surprisingly strong foamability. The shampoo composition gave hair and scalp a very clean feeling in-shower, and a soft, smooth after-feeling. A healthy shine was also observed.

Examples with coco-caprylate and isononyl isononanoate (low-density silicone replacement esters) rather than amla oil resulted in instability of the formula (i.e., separation). Examples with Sodium cocoyl isethionate pellets rather than Sodium cocoyl isethionate powder resulted in reduced dissolution in solvent. Examples without any emollient (e.g., Glyceryl caprylate /caprate) resulted in increased hardness and reduced conditioning property of the final product. Experimentation revealed a negative impact of water removal on foamability, resulting in a need to increase the amount of surfactant as in a concentrate form.

### Example 1D: Shampoo Compositions

In one embodiment, the Shampoo base may be modified to offer better moisturizing, shine, silkiness and soft / smooth after-feel without the use of silicon.

| | %wt |
|---|---|
| Aloe Vera Gel | 9.0000 |
| Glycerin | 14.0000 |
| Sodium Lauryl Glutamate | 20.0000 |
| Dead Sea Salt Coarse | 0.5000 |
| Glycerin | 18.0000 |
| Aloe Vera Gel | 6.0000 |
| Propanediol | 12.0000 |
| Sodium Cocoyl Isothionate | 10.0000 |
| Coco Caprylate/Caprate (and) Triehptanoin (and) C9-C12 Alkane (and) Dilinoleic Acid/Butanediol Copolymer (and) Castor Oil/IPDI Copolymer | 2.0000 |
| Polyglyceryl-3 Stearate, Cetearyl Alcohol, PCA Glyceryl Oleate, Glyceryl Caprylate | 1.5000 |
| PCA glyceryl oleate | 2.0000 |
| Algea oil | 0.5000 |
| Polyglyceryl-3 Betainate Acetate | 1.5000 |
| Essential Oil medley | 3.0000 |

### Example 2: Body Wash Composition

An implementation of the shampoo paste composition is presented herein as an illustration of the application of the cosmetic paste composition as a body wash paste composition.

| | Example 2 |
|---|---|
| Ingredient | w/w% |
| Sodium Cocoyl Isothionate | 14 |
| Sodium Lauroyl Glutamate | 19 |
| Glycerin | 37.7 |
| Aloe Vera Gel | 17.8 |
| Propanediol | 2.4 |
| Lysolecithin (And) Sclerotium Gum (And) Xanthan Gum (And) Pullulan | 0.6 |
| Glyceryl Caprylate/Caprate | 2 |
| Dead Sea Salt | 0.5 |
| Polyglyceryl-3 Betainate Acetate | 3 |
| Essential Oil Blend | 3 |
| TOTAL | 100 |
| Viscosity (at 40C) (cP) | 5000-30000 |
| Consistency at room temperature | Thick paste |
| pH | 5.5-6.5 |
| Specific gravity | 0.80-1.1 |
| Solid wt% | 70.00-85.00 |
| Colour | White |
| Textu re | Smooth paste |
| Scent | Citrus |
| Foamability | High |
| Cleansing | High |
| Conditioning | High |

Preparation of Example 2 was as follows:

1. Phase A: Dead Sea salt was completely dissolved in a mixture of glycerin and aloe vera (“solvent”) at approximately 55° C. Approximately half of the aloe vera and approximately half of the glycerin were used in this phase. The primary surfactant was then mixed and softened in the solvent while maintaining 55° C. temperature.

2. Phase B: The remaining glycerin and aloe vera were combined at 55° C. The secondary surfactant was then mixed and softened in the solvent while maintaining the temperature at 55° C.

3. Phases A and B were combined at 55° C. until the mixture was smooth (“main mix”).

4. The propanediol and Lysolecithin mix were combined at room temperature, then added to the main mix at 55° C. and mixed until uniform consistency.

5. The GCC, Polyglyceryl-3 Betainate Acetate, and essential oils were combined at room temperature, then added to the main mix at 55° C. until uniform consistency.

The body wash composition according to Example 2 had a homogenous and paste-like texture that was easily removed from the application tube and malleable in the user’s hands. The body wash composition activated upon addition of water and shear stress and resulted in a rich, creamy lather with surprisingly strong foamability. The body wash composition removed all visible dirt and gave skin a soft, hydrated after-feel. The high concentration of skincare ingredients such as aloe vera, glycerin and betaine left skin with a noticeably moisturized after feel the more they were worked into the skin when showering despite rinsing off.

### Example 3: Shampoo Composition Comparison to Leading Sulphate Free Liquid

In order to obtain and retain a customer base, the user experience must be considered. It is desirable that the cosmetic properties of the concentrate paste shampoo to exceed expectations of consumers. Much of the population uses a liquid shampoo to clean their hair. A qualitive study comparing the concentrate paste shampoo to the leading sulphate-free liquid shampoo was conducted.

A sample population of 11 users evaluated the ease of use and various aspects of the cosmetic and sensory experience of the concentrate paste shampoo and the leading sulfate-free liquid shampoo. Properties evaluated included ease of dosing, ease of application, product loss, sensorial experience, foaming/lather, ease of rinsing, wet combing, dry combing, smoothing, volume, and softness. Each property was rated by the participants on a scale of 1 to 5. The scores provided by each of the participants were averaged to arrive at the average scores below. The results of the study are summarized in the table below:

| Study Question | Concentrate Paste prepared in accordance to Example 1A | Leading Sulphate Free Liquid Shampoo |
|---|---|---|
| Ease of dosing: Did you get the right amount of shampoo? | 4.0 | 3.8 |
| Ease of application: Was it easy to apply to your hands and/or hair? | 3.7 | 4.5 |
| Product loss: Did you lose product through application and/or storage? | 3.6 | 4.1 |
| (1 is loss, 5 is no loss) | | |
| Sensorial Experience: Did you have a good sensorial experience? | 4.6 | 3.4 |
| Foaming / lather: How well did the product lather? | 4.2 | 4.2 |
| Ease of rinsing: How well did the product rinse out? | 4.6 | 4.4 |
| Wet combing: Was it easy to comb wet hair? | 3.9 | 3.5 |
| Dry combing: Was it easy to comb dry hair? | 4.1 | 3.5 |
| Smoothing: Was your hair smooth and de-frizzed? | 3.8 | 3.1 |
| Volume: Did your hair feel voluminous/lightweight? | 4.3 | 3.7 |
| Softness: Was your hair soft? | 4.6 | 3.5 |
| Total | 49.7 | 45.6 |

As can be seen from the results, the overall performance of the concentrate paste was higher (total score of 49.7 points) than that of the leading sulphate free liquid shampoo (total score of 45.6 points). According to the results, the concentrate paste matched or out preformed the leading sulphate free liquid shampoo in all qualitative properties with the exception of product loss and ease of application since liquid is the form people are most used to. Most notably, users noticed very high performance in the categories of softness, sensorial experience, and volume. While some to the ratings relating to ease of application and product lost were lower than that of the leading sulphate free liquid shampoo, it was found that once a user developed an application technique ease of application and perceived waste decreased.

### Example 4: Shampoo Composition Comparison to Leading Sulphate Free Solid Shampoos

A further study was conducted to compare the cosmetic, sensory and user experience of the paste composition to that of the leading sulphate free solid shampoos. A qualitive study comparing the concentrate paste shampoo to the leading sulphate-free powder shampoo and leading sulphate-free bar shampoo was conducted. A sample population of 11 users evaluated the ease of use and various aspects of the cosmetic and sensory experience of the concentrate paste shampoo and the leading sulfate-free solid shampoos. Properties evaluated included ease of dosing, ease of application, product loss, sensorial experience, foaming/lather, ease of rinsing, wet combing, dry combing, smoothing, volume, softness, and shine. Each property was rated by the participants on a scale of 1 to 5. The scores provided by each of the participants were averaged to arrive at the average scores below. The results of the study are summarized in the table below:

| Study Question | Concentrate Paste prepared in accordance to Example 1A | Leading Sulphate free Powder | Leading Sulphate Free Bar |
|---|---|---|---|
| Ease of dosing Did you get the right amount of shampoo? | 4.5 | 2.5 | 3.6 |
| Ease of application Was it easy to apply to your hands and/or hair? | 4.2 | 2.5 | 3.3 |
| Product loss Did you lose product through application and/or storage? (1 is loss, 5 is no loss) | 4.6 | 2.5 | 3.8 |
| Sensorial Experience Did you have a good sensorial | 5.0 | 2.3 | 2.5 |
| experience? | | | |
| Foaming / lather How well did the product lather? | 4.7 | 3.1 | 3.5 |
| Ease of rinsing How well did the product rinse out? | 4.6 | 3.4 | 4.0 |
| Wet combing: Was it easy to comb wet hair? | 3.5 | 2.6 | 2.8 |
| Dry combing: Was it easy to comb dry hair? | 4.3 | 3.2 | 3.6 |
| Smoothing: Was your hair smooth and de-frizzed? | 3.9 | 3.3 | 3.4 |
| Volume: Did your hair feel voluminous/lightweight? | 4.9 | 3.3 | 3.5 |
| Softness: Was your hair soft? | 4.5 | 3.3 | 3.6 |
| Shine: Was your hair shiny? | 4.6 | 3.3 | 3.3 |
| Total | 53.4 | 35.1 | 40.9 |

Most notably, the paste shampoo outperformed the leading sulphate free powder and leading sulphate free bar shampoos (total scores of 53.4, 35.1 and 40.9, respectively). Furthermore, the test group reported higher performance in all areas tested when compared to the powder and bar samples. Of particular note, the paste shampoo was rated a 5 in for sensorial experience and a 4.7 in lather/foaming. Both of these properties are historically difficult to achieve with sulphate free products.

### Examples 5A-E: Conditioner Compositions

Different formulations of the present disclosure are presented herein as illustrations of various conditioner paste compositions.

| | Example 5A | Example 5B | Example 5C | Example 5D | Example 5E |
|---|---|---|---|---|---|
| Ingredient | w/w% | w/w% | w/w% | w/w% | w/w% |
| Sodium Cocoyl Isethionate | 4 | 6.5 | 5.8 | 4.8 | 4.50 |
| Glyceryl Caprylate/Caprate | 4 | 6 | 4 | 4 | 4.00 |
| Caesalpinia Spinosa Gum | 1 | | | | 1.00 |
| Jojoba Esters | 1.5 | 2.5 | 4.8 | 2.8 | 3.00 |
| Lysolecithin (And) Sclerotium Gum (And) Xanthan Gum (And) Pullulan | | 1.1 | | | |
| Sodium Carboxymethyl Starch | | 6 | | | |
| Xanthan Gum (And) Lecithin (And) Sclerotium Gum (And) Pullulan | | | 1 | 1 | |
| Glycerin | 61.8 | 52.9 | 61.8 | 61.8 | 58.10 |
| Aloe Vera | 2 | 4.2 | 2.6 | 5.6 | 2.00 |
| Brassicyl Isoleucinate Esylate (And) Brassica Alcohol | 2 | 1 | 10 | 8 | 2 |
| Polyglyceryl-3 Stearate, Cetearyl Alcohol, PCA Glyceryl Oleate, Glyceryl Caprylate | 3 | 4.5 | | | 3.00 |
| PCA Glyceryl Oleate | 3 | | | | 3.50 |
| Polyglyceryl-3 Betainate Acetate | 1.3 | | | | 1.30 |
| Hydrolyzed Hyaluronic Acid/ Lactic Acid/ Sodium Hyaluronate | 0.1 | | | | 0.10 |
| Arachidyl/Behenyl Betainate Esylate (And) Arachidyl/Behenyl Alcohol | 8 | | 10 | 8 | 10.00 |
| Lauryl/Myristyl Polyricinoleate (And) Glycerin | 2 | 2.2 | | | 2.00 |
| Behentrimonium Chloride | | 2.6 | | | |
| Coco-Caprylate | | 5 | | | |
| Distearyldimonium Chloride | | | | 4 | |
| Algeo Oil | 2 | | | | 2.00 |
| Essential Oil Medley | 3.8 | 3.5 | | | 3.00 |
| MCT Oil | | 1 | | | |
| Coconut Oil | | 0.1 | | | |
| Shea Butter | | 0.1 | | | |
| Amla Oil | | 0.8 | | | |
| Dead Sea Salt Coarse | 0.5 | | | | 0.5 |
| TOTAL | 100 | 100 | 100 | 100 | |
| Viscosity (at 40C) (cP) | 20,000-35,000 | 50,000-60,000 | n/a | n/a | |
| Consistency at room temperature | Thick paste | Thick paste | Thick paste | Thick paste | |
| pH | 3.5-4.5 | 4.50-5.00 | 2.76-3.38 | 2.81-3.43 | |
| Specific gravity | 0.85-0.98 | | 0.99-1.22 | 0.95-1.17 | |
| Solid wt% | 90.00-100.00 | 90.00-100.00 | | | |
| Colour | White | White | White | White | |
| Textu re | Soft paste | Oily paste | Soft paste | Soft paste | |
| Scent | Citrus | Citrus | Neutral | Neutral | |
| Conditioning | High | Medium-high | High | High | |
| Slip | High | Medium | High | High | |

Preparation of Example 5A was as follows:

1. Phase A: The surfactant was combined at room temperature with approximately half the glycerin and mixed until uniform.

2. Phase B: The Hyaluronic Acid was combined at room temperature with the aloe vera and dead sea salt coarse and mixed until uniform.

3. Phase C: The caesalpinia spinosa gum was combined at room temperature with the remaining glycerin and mixed until uniform.

4. Phases B and C were added to the main mix. The main mix was then mixed slowly, maintaining the temperature, until homogeneous.

5. Phase D: The remaining conditioning agents were combined, and mixed slowly until all waxes were melted.

6. Phase E: The GCC and essential oil were combined, then quickly mixed into Phase D while maintaining 80° C.

7. The main mix was slowly added to Phase D while mixing and maintaining 80° C.

8. While homogenizing, the mixture was maintained at 80° C. for 20-30 minutes, then the temperature was reduced to 60° C. for 2-4 hours, then finally reduced to 50° C. for 5-10 minutes. The mixture was then allowed to cool to room temperature.

The conditioner composition according to Example 5A had a homogenous and paste-like texture that was easily removed from the application tube and malleable in the user’s hands. The conditioner composition activated upon addition of water and shear stress and resulted in a thick, creamy liquid that was capable of coating the user’s hair. The conditioner composition had a surprisingly strong detangling effect and gave hair a silky feeling in-shower, and a soft, hydrated after-feel.

An attempt to reduce the viscosity by adding a cationic and quaternary-based conditioning agent resulted in deficient rinsing capacity. An attempt to overcome excessive adhesion by substituting waxy lipid molecules with anhydrous actives resulted in a viscosity below threshold. An attempt to overcome instability by using a cationic surfactant, for example, PCA Ethyl Cocoyl Arginate, or an amphoteric surfactant, for example, cocamidopropyl betaine, rather than an anionic surfactant, for example, Sodium Cocoyl Isethionate, failed to resolve the issue, implying that the charge on the surfactant was not responsible for the instability.

### Example 6: Hair Mask Composition

An implementation of the conditioner paste composition is presented herein as an illustration of the application of the cosmetic paste composition as a hair mask paste composition. Preparation of the hair mask composition was as follows:

| | Example 6a | Example 6b |
|---|---|---|
| Ingredient | w/w% | w/w% |
| Polyglyceryl-3 Stearate,Cetearyl Alcohol, PCA Glyceryl Oleate, Glyceryl Caprylate | | 5.1 |
| Behentrimonium Methosulfate, Cetearyl Alcohol | 10 | 8 |
| Brassica Alcohol, Brassicyl Valinate Esylate | 10 | 9 |
| Candelilla Wax | 1 | |
| Lauryl/ Myristyl Polyricinoleate, Glycerin | 5 | |
| Ricinus Communis (Castor) Seed Oil, Camellia Japonica Seed Oil, Camellia Sinensis Seed Oil, Camellia Japonica Flower Extract, Camellia Japonica Seed Extract | 2 | 1 |
| Crambe Abyssinica Seed Oil, Phytosterols, Oleyl Alcohol | 3 | 3 |
| MCT Oil (Caprylic/Capric Triglyceride) | 15 | 8.8 |
| Coconut Oil (Cocos Nucifera (Coconut) Oil) | 10 | 5.2 |
| Squalane | 12 | |
| Simmondsia Chinensis (Jojoba) Seed Oil | 5 | 7 |
| Hydrogenated Ethylhexyl Olivate (and) Hydrogenated Olive Oil Unsaponifiables | 10 | |
| Everist Essential Oil Medley | 2 | 2 |
| Sodium Stearoyl Glutamate | 2 | |
| Modified Corn Starch | 5 | |
| Tocopherol | 2 | 1 |
| Dextrin Palmitate | | 1.1 |
| Polyglyceryl-4 Olivate/Polyricinoleate | | 10.7 |
| Glycerin RSPO | | 24.4 |
| Aloe Barbadensis Juice | | 7.2 |
| Sucrose Stearate, Sucrose Palmitate | | 1.7 |
| Xanthan Gum, Lecithin, Sclerotium Gum, Pullulan | | 0.4 |
| Tocopherol | | 1 |

While there was a plethora of challenges associated with the development of the hair mask composition, most notably were colour change and oil separation, as well as oil/ wax residue left on hair.

Some of the trialed formulations changed colour at 50° C. This ranged between orangish-brown to a dark brown colour. The time in which colour change was observed varied, but always deepened over time. Since orangish-brown or dark brown would be considered an undesirable an unappealing colour for a conditioner, a plurality of potential ingredients had to be eliminated from the formulation to avoid colour change.

Challenges in regard to the stability of the hair mask also needed to be addressed. In many of the trials, the oils separated out of the formulation at room or high temperatures. A primary cause for this result is the carrying polarity of the oils, not allowing them to mix and fully incorporate into the formula. In solid anhydrous products, waxes are incorporated to provide structural stability, but also to prevent syneresis (sweating) by creating a crystal network, trapping the liquid oil molecules. Due to the nature of the product being softer in texture, other methods are required to create the network that prevents syneresis. As previously discussed, oil gellants help combat this problem.

Numerous specific details are set forth in order to provide a thorough understanding of the examples described herein. However, it will be understood by those of ordinary skill in the art that the examples described herein may be practiced without these specific details. In other instances, well-known methods, procedures, and components have not been described in detail so as not to obscure the examples described herein. Also, the description is not to be considered as limiting the scope of the examples described herein.

It will be appreciated that the examples and corresponding diagrams used herein are for illustrative purposes only. Different configurations and terminology can be used without departing from the principles expressed herein. For instance, components and modules can be added, deleted, modified, or arranged with differing connections without departing from these principles.

The specific dimensions in the figures are for illustration only and other suitable dimensions employed in accordance with this disclosure will also work in respect of other embodiments.

Although the above principles have been described with reference to certain specific examples, various modifications thereof will be apparent to those skilled in the art as outlined in the appended claims.

| Ingredient | Molecular Structures |
|---|---|
| Shampoo Ingredients or Alternative Ingredients | |
| Glycerin | 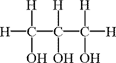 |
| Sodium Lauroyl Glutamate | 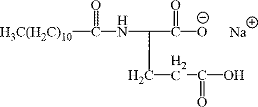 |
| Glycerin | 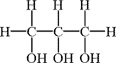 |
| Aloe Barbadensis Leaf Juice | |
| Sodium Cocoyl Isethionate | 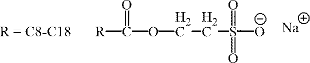 |
| Propanediol | 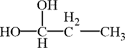 |
| Lysolecithin | 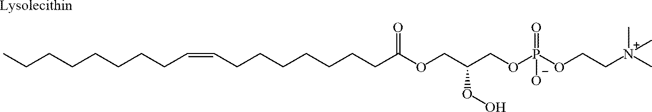 |
| Pullulan | 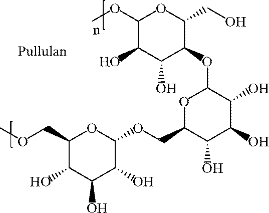 |
| Sclerotium Gum | 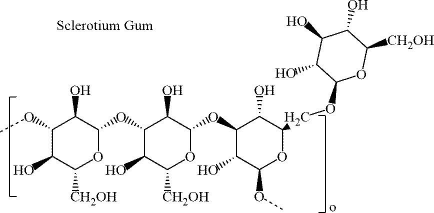 |
| Xanthan Gum | 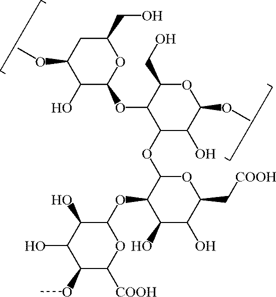 |
| Glyceryl Caprylate/Caprate | 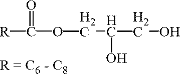 |
| Brassica Campestris (Rapeseed) Seed Oil (and) Sesamum Indicum (Sesame) Seed Oil (and) Emblica Officinalis Fruit Extract | |
| Primary Surfactant or Alternative Primary Surfactant Examples | |
| Sodium Cocoyl Glutamate | 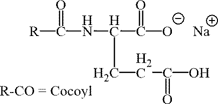 |
| Sodium Cocoyl Glycinate | 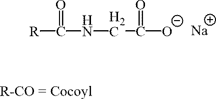 |
| Sodium Cocoyl Alaninate (and) Water | 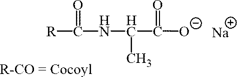 |
| Sodium Myristoyl Glutamate | 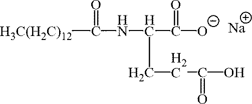 |
| Sodium Stearoyl Glutamate and Sodium Cocoyl Glutamate | 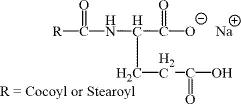 |
| Sodium Cocoyl Threoninate (and) Disodium Cocoyl Glutamate (and) Sodium Cocoyl Glutamate (and) Water | 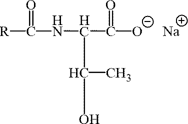 |
| Sodium Cocoyl Threoninate (and) Disodium Cocoyl Glutamate (and) Sodium Cocoyl Glutamate (and) Water | 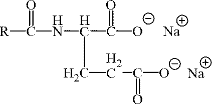 |
| Sodium Lauroyl Sarcosinate | 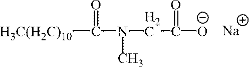 |
| Sodium Myristoyl Sarcosinate | 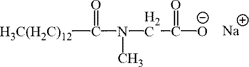 |
| Sodium Cocoyl Sarcosinate | 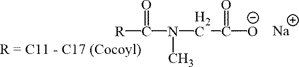 |
| Disodium Laureth Sulfosuccinate | 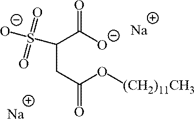 |
| Disodium Cocoyl Glutamate | 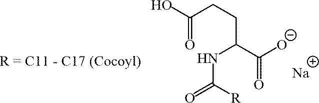 |
| Potassium Cocoyl Glutamate | 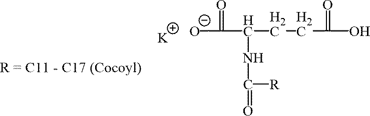 |
| Potassium Cocoyl Glycinate | 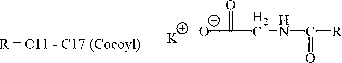 |
| Potassium Myristoyl Glutamate | 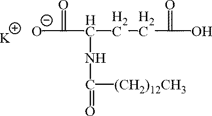 |
| Potassium Lauroyl Glutamate | 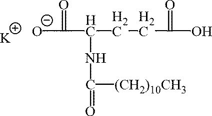 |
| Potassium Lauroyl Sarcosinate | 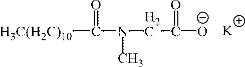 |
| Potassium Cocoate | 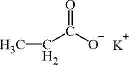 |
| TEA-Cocoyl Glutamate | 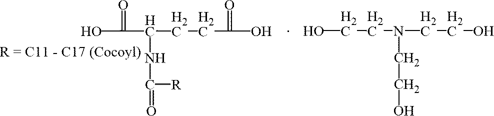 |
| TEA-Cocoyl Alaninate | 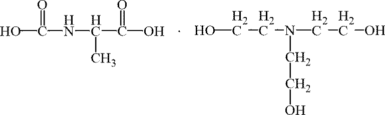 |
| TEA-Lauroyl Glutamate | 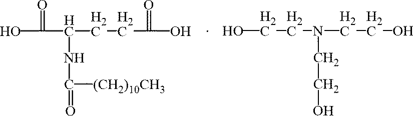 |
| TEA-Lauroyl Sarcosinate | 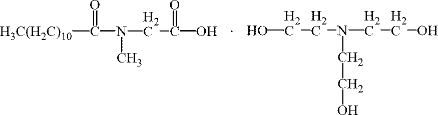 |
| Sodium Stearoyl Glutamate | 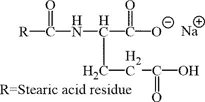 |
| Examples of Co-Surfactants or Alternative Co-Surfactants | |
| Sodium Lauroyl Glycinate | 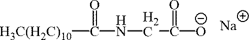 |
| Sodium Lauroyl Lactylate | 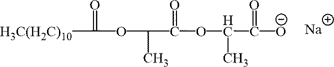 |
| Sodium Lauroyl Isethionate | 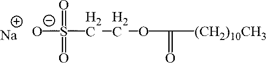 |
| Sodium Lauroyl Methyl Isethionate | 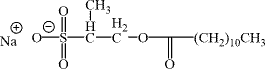 |
| Sodium Methyl Cocoyl Taurate | 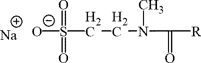 |
| Sodium Cocoyl Methyl Isethionate | 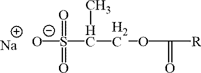 |
| Sodium Methyl Lauroyl Taurate | 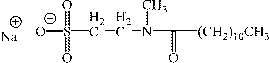 |
| Sodium Cocoyl Glycinate | 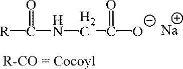 |
| Sucrose Stearate (and) Sucrose Palmltate | 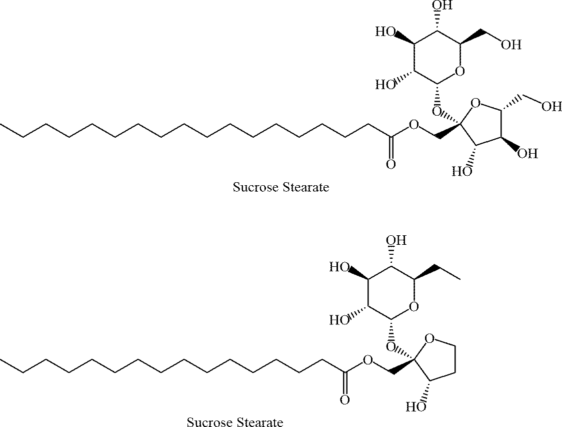 |
| Examples of Gelling Agent or Alternative Gelling Agents | |
| Chondrus Crispus Powder | 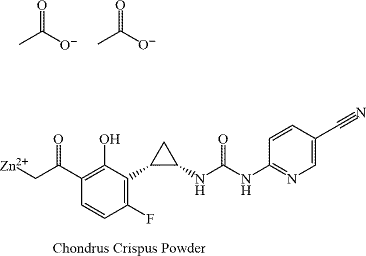 |
| Glucose | 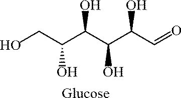 |
| Cellulose Gum | 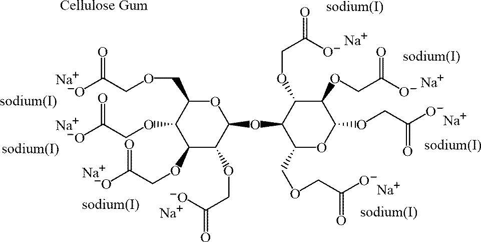 |
| Ceratonia Siliqua (Carob) Gum | 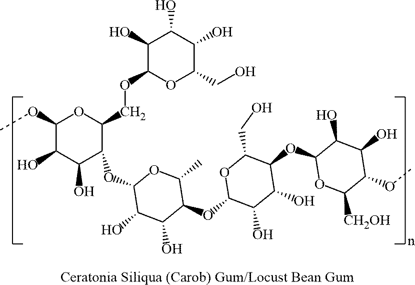 |
| Acacia Senegal Gum (and) Xanthan Gum | 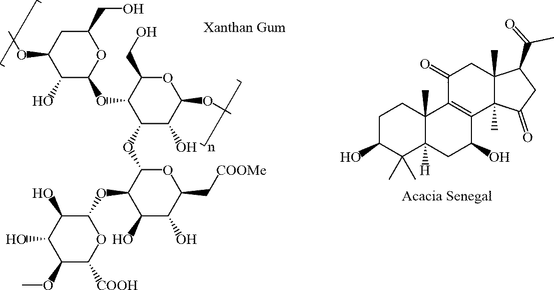 |
| Cyamopsis Tetragonoloba (Guar) Gum | 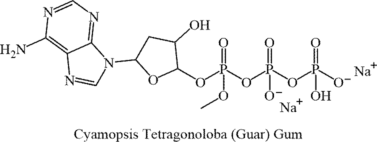 |
| Algin | 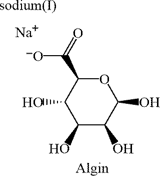 |
| Ceratonia Siliqua (Carob) Gum | 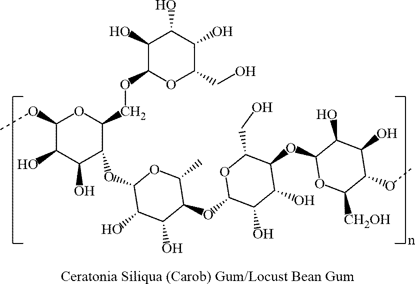 |
| Caesalpinia Spinosa (Tara) Gum | 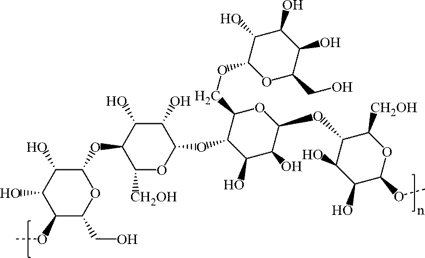 |
| Xanthan Gum (and) Ceratonia Siliqua (Carob) Gum | 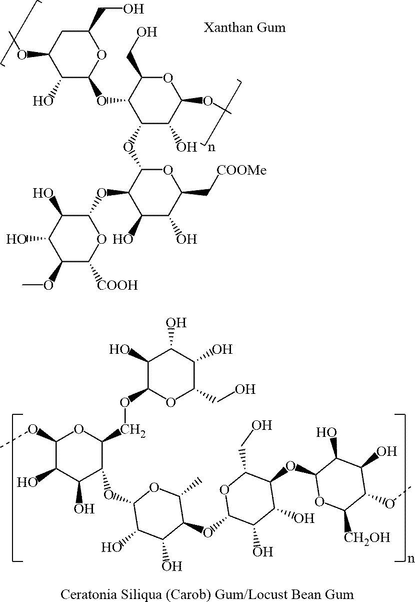 |
| Cellulose Gum | 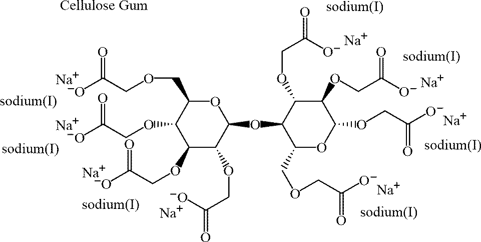 |
| Algin | 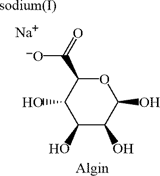 |
| Beta-Glucan | 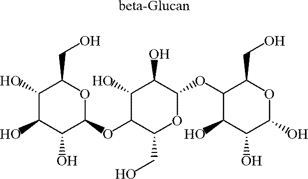 |
| Sclerotium Gum | 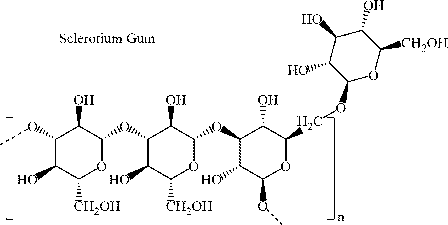 |
| Lecithin | 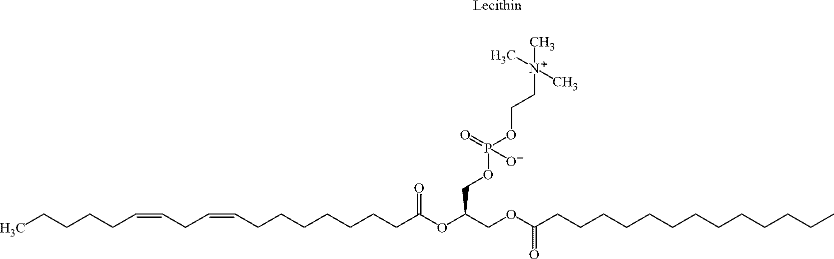 |
| Xantham Gum | 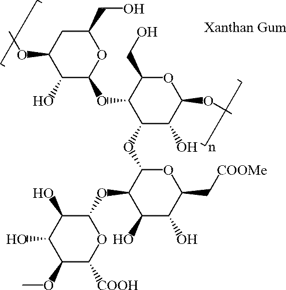 |
| Pullulan | 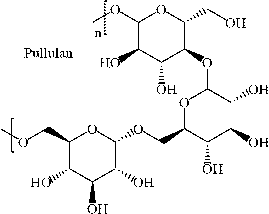 |
| Sclerotium Gum | 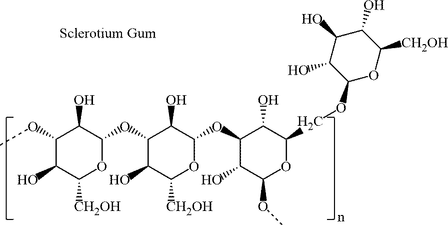 |
| Hydroxypropyl Methylcellulose | 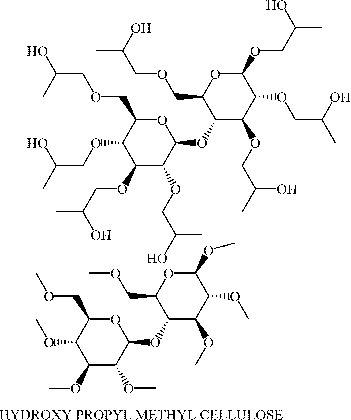 |
| Cellulose Gum | 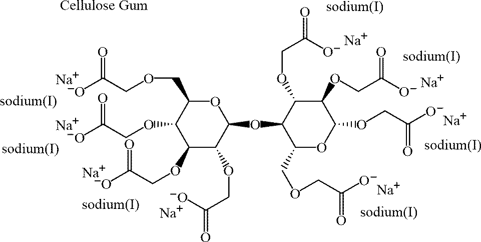 |
| Hydroxyethylcellulose | 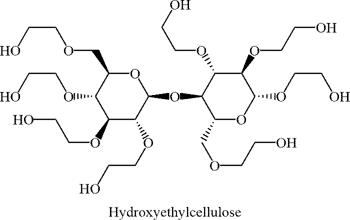 |
| Microcrystalline Cellulose | 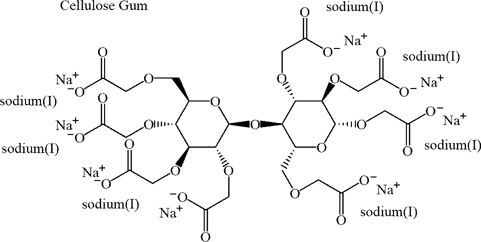 |
| Caprylic/Capric Triglyceride (and) Polyurethane-79 | 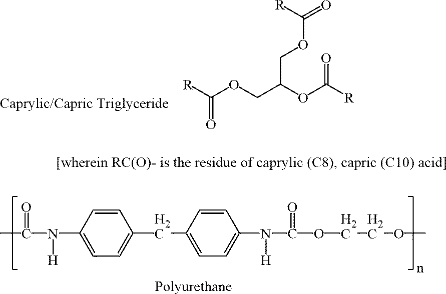 |
| Examples of Alternative Solvents | |
| Butylene Glycol | 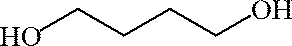 |
| Pentylene Glycol | 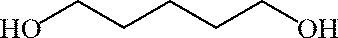 |
| 1,2-Hexanediol | 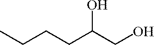 |
| Sorbitol | 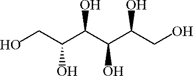 [SMILES: OC[C@@H](O)[C@@H](O)[C@H](O)[C@@H](O)CO] |
| Hexylene Glycol | 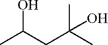 |
| Dipropylene Glycol | 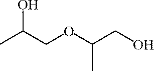 |
| Propylene Glycol | 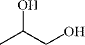 |
| Methylpropanediol | 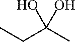 |
| 1,5-Pentanediol | 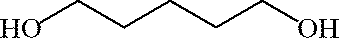 |
| Phenoxypropanediol | 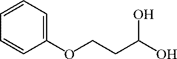 [SMILES: OC(O)CCOc1ccccc1] |
| Phenoxyisopropanol | 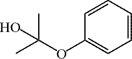 |
| Examples of GCC Ingredients or Alternatives | |
| Glyceryl Abietate | 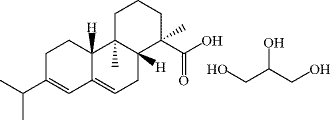 |
| Glyceryl Laurate | 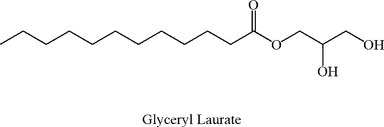 |
| Glyceryl Isostearate | 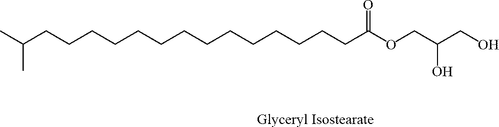 |
| Glyceryl Linoleate | 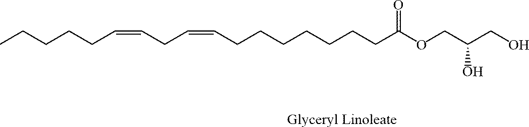 |
| Glyceryl Linolenate | 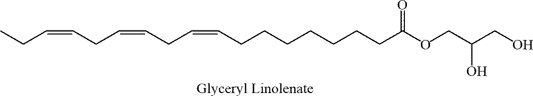 |
| Glyceryl Oleate | 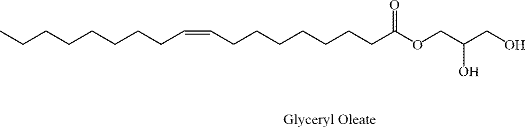 |
| Isoamyl Caprate-Caprylate | 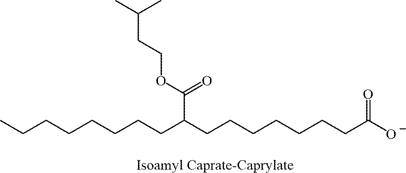 |
| Coco-Caprylate/Caprate (and) Hydrogenated Olive Oil Unsaponifiables | 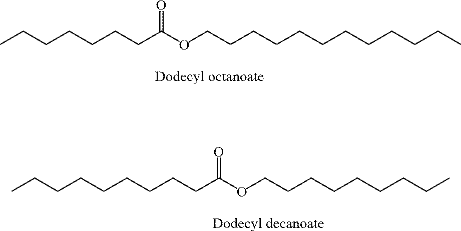 |
| Caprylic/Capric Triglyceride | 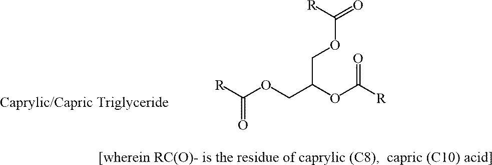 |
| Coco-Caprylate | 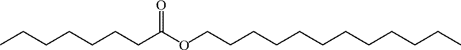 [SMILES: CCCCCCCCCCCCOC(=O)CCCCCCC] |
| Propylene Glycol Dicaprate | 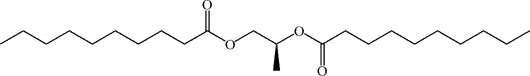 [SMILES: CCCCCCCCCC(=O)OC[C@H](C)OC(=O)CCCCCCCCC] |
| Propanediol Dicaprylate/Caprate | 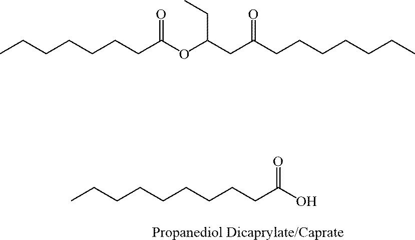 |
| Examples of Other Possible Ingredients or Alternative Ingredients | |
| Sodium Lauriminodipropionate | 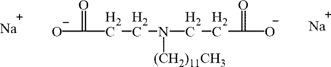 |
| Lauryl Dimethylamine Oxide | 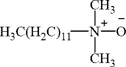 |
| Myristyl/Cetyl Dimethylamine Oxide | 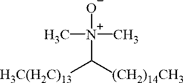 |
| Lauramine Oxide | 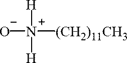 |
| Lauramine Oxide | 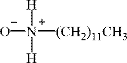 |
| Myristamine Oxide | 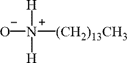 |

1. A shampoo composition in the form of a paste, the composition comprising:
at least one surfactant in an amount of 15 wt% to about 48 wt%;
at least one thickener in an amount of about 0 wt% to about 10 wt%;
at least one organic solvent in an amount of 25 wt% to about 60 wt%; and
at least one emollient in an amount of 0 wt% to 30 wt%;
wherein the shampoo composition is free of, or substantially free of, water.

2. The shampoo composition of claim 1, wherein all or substantially all of the ingredients are plant-derived.

3. The shampoo composition of claim 2, wherein the at least one surfactant is an anionic surfactant.

4. The shampoo composition of claim 3, wherein the at least one organic solvent is a polyol.

5. The shampoo composition of claim 4, wherein the at least one organic solvent is glycerin.

6. The shampoo composition of claim 5, wherein the at least one organic solvent is glycerin and aloe vera gel.

7. The shampoo composition of claim 6, wherein the at least one surfactant is sodium cocoyl isethionate and/or sodium lauroyl glutamate.

8. The shampoo composition of claim 7, wherein the at least one thickener is lysolecithin, sclerotium gum, xanthan gum, and/or pullulan.

9. The shampoo composition of claim 7 wherein the at least one emollient is glyceryl caprylate/caprate.

10. The shampoo composition of claim 9 further comprising propanediol.

11. The shampoo composition of claim 10 further comprising Amla oil.

12. The shampoo composition of claim 11 further comprising at least one conditioning agent.

13. The shampoo composition of claim 12, wherein the at least one conditioning agent is polyglyceryl-3 betainate acetate.

14. The shampoo composition of claim 13, wherein the viscosity of the composition is 15,000-65,000 cps at 40° C. in production, and the composition is a thick paste at room temperature.

15. A conditioner composition in the form of a paste, the composition comprising:
at least one thickener in an amount of about 0.1 wt% to about 8 wt%;
at least one organic solvent and humectant in an amount of about 20 wt% to about 70 wt%;
at least one emulsifier and rheology modifier in an amount of about 1.5 wt% to about 10 wt%;
at least one emollient in an amount of about 2 wt% to about 35 wt%; and
at least one conditioning agent in an amount of 5 wt% to about 40 wt%; wherein the conditioner composition is free of, or substantially free of, water.

16. The conditioner composition of claim 15, wherein the at least one thickener is caesalpinia spinosa gum.

17. The conditioner composition of claim 16, wherein the at least one organic solvent and humectant is a polyol.

18. The conditioner composition of claim 17, wherein the at least one organic solvent is glycerin.

19. The conditioner composition of claim 18, wherein the at least one organic solvent is glycerin, aloe vera gel, sodium hyaluronate, hydrolyzed hyaluronic acid, phenoxyethanol, and/or lactic acid.

20. The conditioner composition of claim 19, wherein the at least one emulsifier and rheology modifier is jojoba esters.
